(19) United States
(12) Patent Application Publication
(10) Pub. No.: US 2025/0288286 A1
(43) Pub. Date: Sep. 18, 2025

(54) SOFT TISSUE ANCHORS AND METHOD

(71) Applicant: EDWARDS LIFESCIENCES CORPORATION, Irvine, CA (US)

(72) Inventors: YUVAL KASHER, Kfar Shmuel (IL); Zachary Christopher Berwick, Tustin, CA (US); Mazen Saadi Albaghdadi, Irvine, CA (US); Allen Jeong Keel, Irvine, CA (US); Atiya Makhdoom Ahmed, Irvine, CA (US)

(73) Assignee: EDWARDS LIFESCIENCES CORPORATION, Irvine, CA (US)

(21) Appl. No.: 19/222,909

(22) Filed: May 29, 2025

Related U.S. Application Data
(63) Continuation of application No. PCT/US2023/081386, filed on Nov. 28, 2023.
(60) Provisional application No. 63/387,921, filed on Dec. 16, 2022.

(51) Int. Cl. A61B 17/00; A61B 17/32

(52) U.S. Cl. CPC A61B 17/00234; A61B 17/32; A61B 2017/00243; A61B 2017/00305; A61B 2017/00336; A61B 2017/00349; A61B 2017/00557

(57) ABSTRACT

Provided is a device for use in medical procedures comprising an elongated body with one or more anchor devices associated with a distal end of the elongated body, whereby the distal end of the elongated body can operably be connected to soft tissue of a subject as part of a medical procedure. The elongated body, in some embodiments, is used as a guide to a medical device, such as a pericardial transection device used for forming cuts in pericardial tissue of a heart, whereby the elongated body provides for guidance and stability when the pericardial transection device is in use. Also provided are various soft tissue anchors, medical kits, and procedures for anchoring an elongated body in soft tissue via use of an anchor device, as well as use of an anchored elongated body in medical procedures such as transection procedures associated with the pericardium.

### PRIORITY CLAIM UNDER 35 U.S.C. § 119

This application is a continuation application of International Patent Application No. PCT/US2023/081386, filed Nov. 28, 2023, which claims the benefit of U.S. Provisional Application No. 63/387,921, filed on Dec. 16, 2022. Each of the foregoing patent applications are hereby incorporated by reference in their entireties.

### TECHNICAL FIELD

This disclosure is directed to an anchoring device for use with soft tissue, e.g., a pericardial layer, fibrous layer, and/or adipose tissue, for example, to thereby provide an anchor support for a medical device when in use. Methods of anchoring a medical device, such as a pericardial transection device in soft tissue are disclosed, such as for example, a guidewire or cannula with an anchor adjacent its distal end for anchoring an end of the guidewire or cannula to soft tissue, where the guidewire or cannula is subsequently used as part of a medical procedure to guide a medical device along a selected path, such as a path for cutting a pericardial layer.

### BACKGROUND

Pericardial restraint is a normal physiologic process that becomes exaggerated, for example, in some patients with heart failure with preserved ejection fraction (HFpEF) and causes the right heart to experience a lower filling volume, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

Provided is a device for use in medical procedures comprising an elongated body with one or more anchor devices associated with a distal end of the elongated body, whereby the distal end of the elongated body can operably be connected to soft tissue of a subject as part of a medical procedure. The elongated body, in some embodiments, is used as a guide to a medical device, such as a pericardial transection device used for forming cuts in pericardial tissue of a heart, whereby the elongated body provides for guidance and stability when the pericardial transection device is in use. Also provided are various soft tissue anchors, medical kits, and procedures for anchoring an elongated body in soft tissue via use of an anchor device, as well as use of an anchored elongated body in medical procedures such as transection procedures associated with the pericardium.

In one embodiment, an elongated body is provided for use in a medical procedure. The elongated body comprises a proximal end and a distal end, and at least one anchor coupled adjacent to the distal end of the elongated body for anchoring the elongated body to soft tissue. The comprises first and second configurations, wherein in the first configuration the anchor is configured to avoid attachment to soft tissue and in the second configuration the anchor is configured to secure itself relative to at least a portion of the soft tissue. A pericardial transection device configured for connection to the elongated body.

In one or further embodiment, the anchor comprises an expandable body configured for transitioning from the first configuration in a compressed state to an expanded state in the second configuration to thereby press the anchor against the soft tissue.

In one or further embodiment, the anchor comprises one of: an inflatable bladder, such that in the second configuration, the bladder is inflated to thereby press the anchor against the soft tissue; one or more collapsible and radially expandible arms for selectively contacting the soft tissue in the second configuration; a helical screw ending in a distal end for inserting the anchor into the soft tissue; or a clamp comprising two or more expandible arms configured to attach to the soft tissue; or one or more protruding barbs configured to attach to the soft tissue.

In one or further embodiment, the elongated body is one of a guidewire or a cannula.

In one or further embodiment, the pericardial transection device is a scalpel, a mechanical cutting device, an electrode, an RF electrode, an electrosurgical device, a reversibly retractable knife blade, or combination of any two or more thereof.

In one or further embodiment, a sheath is provided where the sheath is configured for housing the anchor prior to attaching the anchor to the soft tissue.

In one or further embodiment, at least a portion of the elongated body is sterilized.

In one or further embodiment, a probe is associated with the elongated body, wherein the probe is a nerve stimulating device or a nerve detecting device.

In one embodiment, an elongated body for use in a medical procedure is provided. The elongated body comprises a proximal end and a distal end and at least one anchor coupled adjacent to the distal end of the elongated body for anchoring the elongated body to soft tissue. The anchor of this embodiment has first and second configurations. In the first configuration the anchor is configured to avoid attachment to soft tissue, and in the second configuration the anchor is configured to secure itself relative to at least a portion of the soft tissue.

Various anchor configurations can be configured for use with the elongated body. In one embodiment, the anchor device comprises an expandable body configured for transitioning from the first configuration in a compressed state to an expanded state in the second configuration to thereby press the anchor against the soft tissue. In this or a further embodiment, the anchor device comprises an inflatable bladder, such that in the second configuration, the bladder is inflated to thereby press the anchor against the soft tissue. In this or a further embodiment, the anchor device comprises one or more collapsible and radially expandible arms or struts for selectively contacting the soft tissue in the second configuration. In one embodiment, a bladder covers the radially expandable arms or struts, such that when the arms are expanded, the bladder is expanded to contact the soft tissue.

In one or further embodiments, the anchor device comprises a helical screw ending in a distal end for inserting the anchor into the soft tissue. In this embodiment, the helical screw can be inserted into the soft tissue by rotating the elongated body, or a torsion spring may be associated with the helical screw for causing the helical screw to rotate to thereby contact the soft tissue.

In one or further embodiments, the anchor device comprises a clamp comprising two or more expandible arms configured to attach to the soft tissue. In a first configuration, the expandable arms are in an open disengaged configuration and in a second configuration they are closed relative to each either in a deployed configuration to attach or couple to soft tissue.

In one or further embodiments, the anchor device comprises one or more protruding barbs configured to attach to the soft tissue. In one or further embodiments, the one or more protruding barbs are retractable and deployable for attaching to the soft tissue.

In one or more embodiments, the elongated body is a guidewire, and the anchor is connected to the distal end of the guidewire. In one or more embodiments, the elongated body is a cannula, and the anchor is connected to the distal end of the cannula. In this embodiment, the cannula can include an inner lumen for maintaining the anchor device prior to the anchor device being deployed in a second configuration for connection/attachment to soft tissue. In one or more embodiments, the elongated body is a guidewire or cannula that is steerable, such that once the guidewire or cannula is anchored, the body of the guidewire or cannula can be manipulated to move to a desired location.

In one or further embodiments, the elongated body and anchor device are configured for insertion into a catheter or microcatheter. In some embodiments, the elongated body and the anchor device are configured for insertion into a multi-lumen catheter.

In one or further embodiments, a probe is associated with the elongated body. In one or further embodiments, the probe is a nerve stimulating device or a nerve detecting device.

In one or further embodiments, a sheath is provided that is configured for housing the anchor prior to attaching the anchor to the soft tissue. In one or further embodiments, the sheath comprises one or more lumens in which the anchor device is located in a first configuration and as the anchor device exits the lumen of the sheath, such as when the sheath is slid relative to the anchor device, the anchor device assumes a second configuration for attaching or connecting to soft tissue.

In one or further embodiments, a medical device, such as a pericardial transection device may be configured for connection to the elongated body. In one or further embodiments, the device is a scalpel, a mechanical cutting device, an electrode, an RF electrode, an electrosurgical device, a reversibly retractable knife blade, or combination of any two or more thereof.

In one or further embodiments, at least a portion of the elongated body comprises radiopaque material.

In one or further embodiments, at least a portion of the elongated body is sterilized.

In one or further embodiments, an elongated body for use in a medical procedure is provided where the elongated body has a proximal end and a distal end. One or more anchor devices are coupled adjacent to the distal end of the elongated body for anchoring the elongated body to soft tissue, the anchor having first and second configurations, wherein in the first configuration the anchor is configured to avoid attachment to soft tissue, and in the second configuration the anchor is configured to secure itself relative to at least a portion of the soft tissue. A is provided sheath comprising a lumen for receiving at least the anchor device and allowing the anchor device to move relative to the lumen, such that when the anchor device is located in the lumen, it is in the first configuration and when the anchor is slid out of the lumen, the anchor transitions to the second configuration.

Depending on the embodiment, one or more anchor devices are provided, such as: 1) an anchor device comprising an expandable body configured for transitioning from the first configuration in a compressed state to an expanded state in the second configuration to thereby press the anchor against the soft tissue, 2) an inflatable bladder, such that in the second configuration, the bladder is inflated to thereby press the anchor against the soft tissue, 3) one or more collapsible and radially expandible arms or struts for selectively contacting the soft tissue in the second configuration, where in one embodiment a bladder covers the radially expandable arms or struts, such that when the arms are expanded, the bladder is expanded to contact the soft tissue, 4) a helical screw ending in a distal end for inserting the anchor into the soft tissue, 5) a clamp comprising two or more expandible arms configured to attach to the soft tissue, and/or 6) one or more protruding barbs configured to attach to the soft tissue.

In one or more embodiments, the elongated body is a guidewire, and the anchor is connected to the distal end of the guidewire. In one or more embodiments, the elongated body is a cannula, and the anchor is connected to the distal end of the cannula. In this embodiment, the cannula can include an inner lumen for maintaining the anchor device prior to the anchor device being deployed in a second configuration for connection/attachment to soft tissue.

In one or further embodiments, the elongated body and anchor device are configured for insertion into a catheter or microcatheter. In some embodiments, the elongated body and the anchor device are configured for insertion into a multi-lumen catheter.

In one or further embodiments, a probe is associated with the elongated body. In one or further embodiments, the probe is a nerve stimulating device or a nerve detecting device.

In one or further embodiments, a sheath is provided that is configured for housing the anchor prior to attaching the anchor to the soft tissue. In one or further embodiments, the sheath comprises one or more lumens in which the anchor device is located in a first configuration and as the anchor device exits the lumen of the sheath, such as when the sheath is slid relative to the anchor device, the anchor device assumes a second configuration for attaching or connecting to soft tissue.

In one or further embodiments, a medical device, such as a pericardial transection device may be configured for connection to the elongated body. In one or further embodiments, the device is a scalpel, a mechanical cutting device, an electrode, an RF electrode, an electrosurgical device, a reversibly retractable knife blade, or combination of any two or more thereof.

In one or further embodiments, at least a portion of the elongated body comprises radiopaque material.

In one or further embodiments, at least a portion of the elongated body is sterilized.

In one embodiment, a medical kit is provided comprising: a guidewire comprising an elongated body spanning between a proximal end and a distal end and an anchor coupled adjacent to the distal end for anchoring the guidewire to soft tissue, the anchor having first and second configurations, wherein in the first configuration the anchor is configured to avoid attachment to soft tissue, and in the second configuration the anchor is configured to secure itself relative to at least a portion of the soft tissue. A pericardial transection device is provided and configured for attaching to the elongated body of the guidewire for deployment.

In one or further embodiment, a catheter is provided comprising a lumen, wherein said guidewire is configured for insertion into said lumen of said catheter.

In one or further embodiment, the medical device includes a guide configured for attaching the medical device to the elongated body of said guidewire such that the medical device is capable of movement along the elongated body of said guidewire.

In one or further embodiment, the anchor comprises one of: an inflatable bladder, such that in the second configuration, the bladder is inflated to thereby press the anchor against the soft tissue; one or more collapsible and radially expandible arms for selectively contacting the soft tissue in the second configuration; a helical screw ending in a distal end for inserting the anchor into the soft tissue; a clamp comprising two or more expandible arms configured to attach to the soft tissue; or one or more protruding barbs configured to attach to the soft tissue.

In one or further embodiments, a medical kit is provided that comprises a guidewire comprising an elongated body spanning between a proximal end and a distal end and an anchor device coupled adjacent to the distal end for anchoring the guidewire to soft tissue. The anchor device has first and second configurations, wherein in the first configuration the anchor is configured to avoid attachment to soft tissue, and in the second configuration the anchor is configured to secure itself relative to at least a portion of the soft tissue. The kit comprises a medical device configured for attaching to the elongated body of the guidewire for deployment.

In one or further embodiments, the medical kit comprise a catheter comprising a lumen, wherein the guidewire is configured for insertion into the lumen of the catheter. In one or further embodiments, the anchor device is located in the same lumen as the guidewire or in a second lumen of the catheter.

In one or further embodiments, the medical device includes a guide configured for attaching the medical device to the elongated body of said guidewire such that the medical device is capable of movement along the elongated body of said guidewire.

Depending on the embodiment, one or more anchor devices are provided, such as: 1) an anchor device comprising an expandable body configured for transitioning from the first configuration in a compressed state to an expanded state in the second configuration to thereby press the anchor against the soft tissue, 2) an inflatable bladder, such that in the second configuration, the bladder is inflated to thereby press the anchor against the soft tissue, 3) one or more collapsible and radially expandible arms or struts for selectively contacting the soft tissue in the second configuration, where in one embodiment a bladder covers the radially expandable arms or struts, such that when the arms are expanded, the bladder is expanded to contact the soft tissue, 4) a helical screw ending in a distal end for inserting the anchor into the soft tissue, 5) a clamp comprising two or more expandible arms configured to attach to the soft tissue, and/or 6) one or more protruding barbs configured to attach to the soft tissue.

In one or further embodiments, the medical device is a pericardial transection device. In one or further embodiments, the medical device is a pericardial transection device in the form of a scalpel, a mechanical cutting device, an electrode, an RF electrode, an electrosurgical device, a reversibly retractable knife blade, or combination of any two or more thereof.

In one or further embodiments, the medical kit comprises a probe associated with the catheter, such as a nerve stimulating device or a nerve detecting device.

In one or further embodiments, the at least a portion of the guidewire or the medical device or both comprises radiopaque material.

In one or further embodiments, the guidewire or the medical device or both is sterilized.

In one or further embodiments, there is provided an anchored medical device comprising an elongated body spanning between a proximal end and a distal end. An anchor device is coupled adjacent to the distal end for anchoring the elongated body to soft tissue, the anchor having first and second configurations, wherein in the first configuration the anchor is configured to avoid attachment to soft tissue, and in the second configuration the anchor is configured to secure itself relative to at least a portion of the soft tissue, wherein a portion of the elongated body in a pericardial cavity or a pericardium and the anchor is in the second configuration and is secured to a portion of the pericardium. A pericardial transection device is located in the pericardial cavity and in contact with at least a parietal layer of the pericardium.

In one embodiment, a method is provided for anchoring a device in a pericardium of a subject. The method comprising: presenting an anchor to a pericardial cavity of the pericardium; traversing the anchor within the pericardial cavity; and coupling the anchor with at least a portion of the pericardium.

In one or further embodiment, the method comprises coupling the anchor to one of: epicardial tissue of the pericardium; or the parietal layer.

In one or further embodiment, the method further comprises after the traversing step of entering the parietal layer from the pericardial cavity with the anchor and puncturing through the parietal layer to outside of the pericardium, and said coupling comprises coupling the anchor to an outer layer of the pericardium.

In one or further embodiment, the anchor is attached to a distal end of either a guidewire or cannula, and wherein said traversing comprising advancing the anchor using the guidewire or cannula within the pericardial cavity.

In one or further embodiment, the method further comprising: coupling a pericardial transection device to the elongated body; and advancing the pericardial transection device along the elongated body toward the distal end of the elongated body.

In one or further embodiment, the method comprising: deploying a cutting instrument from the pericardial transection device; traversing the pericardial transection device relative to the elongated body; and cutting at least a portion or pericardial tissue using the pericardial transection device.

In one or further embodiment, the method further comprising applying a force to the elongated body so as to position the pericardial transection device coupled to the elongated body at a selected location in the pericardial cavity.

A method of anchoring a device in a pericardium of a subject is also provided. According to one embodiment, the method comprises presenting an anchor to a pericardial cavity of the pericardium, traversing the anchor within the pericardial cavity, and coupling the anchor with at least a portion of the pericardium. In one or further embodiments, the coupling operation comprises coupling the anchor to at least epicardial tissue of the pericardium. In one or further embodiments, the coupling operation comprises coupling the anchor to the parietal layer.

In one or further embodiments, the method comprises after traversing step, entering the parietal layer from the pericardial cavity with the anchor and coupling the anchor to an outer layer of the pericardium.

In one or further embodiments, the method presenting an anchor to the pericardial cavity comprises puncturing from an outer surface of the pericardium through to the pericardial cavity.

In one or further embodiments, the anchor is attached to a distal end of a guidewire, and wherein said traversing comprising advancing the anchor using the guidewire within the pericardial cavity.

In one or further embodiments, the anchor is attached to a distal end of a catheter, and wherein traversing comprising advancing the anchor using the catheter within the pericardial cavity.

In one or further embodiments, the anchor comprises an expandable body configured for transitioning from the first configuration in a compressed state to an expanded state in the second configuration, and wherein coupling comprises placing the expandable body in an expanded state to thereby press the anchor against the soft tissue.

In one or further embodiments, the anchor comprises an inflatable bladder configured for transitioning from the first configuration in a compressed state to an expanded state in the second configuration, and wherein coupling comprises placing the expandable body in an inflated state to thereby press the anchor against the soft tissue.

In one or further embodiments, the anchor comprises one or more a collapsible and radially expandible arms configured for transitioning from the first configuration in an open state to a closed state in the second configuration, and wherein said coupling comprises placing the expandable body in the closed state to thereby contact the soft tissue.

In one or further embodiments, the anchor comprises a helical screw ending in a distal end for inserting the anchor into the soft tissue, and wherein coupling comprises inserting the helical screw into the soft tissue.

In one or further embodiments, the anchor comprises a clamp comprising two or more expandible arms configured to attach to the soft tissue, and wherein coupling comprises closing the expandible arms to connect the anchor to the soft tissue.

In one or further embodiments, the anchor comprises one or more protruding barbs configured to attach to the soft tissue and wherein coupling comprises bringing the protruding barbs in contact with the soft tissue. In one or further embodiments, the one or more protruding barbs are retractable and deployable for attaching to the soft tissue.

In one or further embodiments, the traversing operation comprises traversing from an entry point to an anchoring point.

In one or further embodiments, the elongated body is a guidewire, and the anchor is connected to the distal end of the guidewire.

In one or further embodiments, the elongated body is a cannula, and the anchor is connected to the distal end of the cannula.

In one or further embodiments, the elongated body is configured for insertion into a catheter or microcatheter. In one or further embodiments, the elongated body is a configured for insertion into a multi-lumen catheter.

In one or further embodiments, the method further comprising coupling a pericardial transection device to the elongated body. In one or further embodiments, the pericardial transection device is a scalpel, a mechanical cutting device, an electrode, an RF electrode, an electrosurgical device, a reversibly retractable knife blade, or combination of any two or more thereof.

In one or further embodiments, the further comprising coupling a pericardial transection device to the elongated body and advancing the pericardial transection device along the elongated body toward the distal end of the elongated body.

In one or further embodiments, the method further comprising deploying a cutting instrument from the pericardial transection device, traversing the pericardial transection device relative to the elongated body, and cutting at least a portion or pericardial tissue using the pericardial transection device.

In one or further embodiments, the method further comprising probing using either one or both of a nerve stimulating device or a nerve detecting device to detect presence of a nerve.

In one or further embodiments, the method further comprising tracking at least a portion of the elongated body via sensing the presence of a radiopaque material.

In one or further embodiments, the method further comprising use of an elongated body, wherein at least a portion of the elongated body is sterilized.

In one or further embodiments, the location of the elongated body can be controlled by for example, placing a backward, longitudinal force on the elongated body, so as to place the elongated body under tension. In this or further embodiments, a medical device, such as a pericardial transection device, can be operatively coupled to the elongated body and advanced along the elongated body to a selected position. The medical device could then be deployed, such as deployment of a blade from a pericardial transection device, and then reversed back along the elongated body for cutting tissue, while the elongated body, under tension, providing a controlled cutting path.

In one further embodiment, the elongated body is a guidewire, such as an extra stiff Lunderquist® guidewire with 0.035 inch Nitinol core. In this embodiment, once the distal end of the guidewire is anchored, the position of the body of the guidewire can be controlled by applying a force to the guidewire, such as a rotation, a longitudinal force, or both to the guidewire. For example, in one embodiment, a lateral trajectory of the guidewire can be achieved by rotation of the guidewire towards the desired direction followed by application of a longitudinal force in a direction toward the anchor. This, in turn, will assist the guidewire to curve into position. In this or further embodiments, a medical device, such as a pericardial transection device, can be operatively coupled to the guidewire body and advanced along the guidewire body to a selected position. The medical device could then be deployed, such as deployment of a blade from a pericardial transection device, and then reversed back along the guidewire body for cutting tissue, while the guidewire body, under tension, providing a controlled cutting path in curved configuration based on the curvature of the guidewire and at the location of the guidewire.

In one or further embodiments, the method further comprising applying one or more of a longitudinal force, torsional force, or a rotational force to said elongated body.

In one or further embodiments, the method further comprising applying a force to the elongated body comprising applying a force such that at least a portion of the elongated body flexes or bends with the pericardial cavity.

In one or further embodiments, the elongated body is a guidewire or cannula that is steerable, wherein the method comprises manipulating the elongated body, via steering, to place the elongated body at a selected position.

In one or further embodiments, the method further comprising deploying a cutting instrument from the pericardial transection device, traversing the pericardial transection device relative to the elongated body, and cutting at least a portion or pericardial tissue using the pericardial transection device.

### BRIEF DESCRIPTION OF THE DRAWINGS

In order to understand and to see how the present disclosure can be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1B of FIG. 1A depicting the serosal, visceral, fibrous layers and adipose tissue of the parietal pericardium, including the pericardial cavity.

FIG. 2 is a depiction of an exemplary soft tissue anchor device deployed relative to a pericardium of the heart depicted in FIGS. 1A-1C, as disclosed and described herein according to one or more embodiments.

FIGS. 3A, 3A1 is a depiction of an anchor device according to one or more embodiments.

FIGS. 3B-3G each is a depiction of an anchor device according to one or more embodiments.

FIGS. 4A-4C are depictions of an anchor device according to one or more embodiments of the present invention in combination with a pericardial transection device according to one or more embodiments.

FIGS. 5A, 5B depict deployment of an anchor device and a pericardial transection device into a pericardial cavity of a human or animal according to one or more embodiments of the present invention to thereby form cuts in pericardial tissue.

FIGS. 6A-6C depict deployment of an anchor device relative to pericardial tissue of a heart according to one or more embodiments.

FIG. 7 is a depiction of an operation to deploy an anchor device and a pericardial transection device according to one or more embodiments into a pericardial cavity and perform incisions or cuts into pericardial tissue of a heart according to one or more embodiments.

FIGS. 8A-8C depict operations according to one or more embodiments of the present invention associated with selected displacement of anchor assembly and pericardial transection device according to one or more embodiments.

FIGS. 9A-9C depict operations according to one or more embodiments of the present invention associated with selected displacement of anchor assembly and pericardial transection device according to one or more embodiments in a pericardial cavity of a patient to make one or more incisions or cuts therein according to one or more embodiments.

FIGS. 10A-10B depict operations according to one or more embodiments of the present invention associated with selected displacement of anchor assembly and pericardial transection device according to one or more embodiments in a pericardial cavity of a patient to make one or more incisions or cuts therein according to one or more embodiments.

### DETAILED DESCRIPTION

The following terms may be used in this disclosure and will have the meaning as provided below.

As used herein the phrase “elongated body” refers to any structure having a body extending between proximal and distal ends suitable for insertion into a body, such as a human or animal, and have a level of flexibility for insertion into, traversal within, and removal from a human or animal body. The below disclosure discloses an elongated body as at least one of a guidewire or a cannula. In some embodiments, the term guidewire is used interchangeably with elongated body. It is understood that any embodiment where a guidewire is discussed, a cannula could be used as opposed to the guidewire. Generally, a cannula would be employed where use of the central bore (i.e., lumen) in the cannula is used as part of the procedure, such as to maintain the disclosed anchor devices.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed adjacent to and including adipose tissue within and outside the pericardial cavity and superficial to the visceral layer of the pericardium.

As used herein the phrase “cutting surface” is inclusive of one or more of an edge of a sharpened blade or the surface of an electrode configured to receive sufficient current or radio frequency energy (RF) to ablate, burn, vaporize or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter or catheter, and the application of a directional force sufficient to cut or separate the tissue, the force being substantially in a direction towards the proximal end of the multi-lumen catheter or catheter, for example, by pulling the multi-lumen catheter or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an end point. An incision length can be linear, non-linear, or a plurality of linear and/or non-linear lengths that intersect or do not intersect about a curved or non-planar surface, such as the heart.

As used herein the phrase “reducing pressure” and “reducing restraint” are inclusive of their ordinary and customary meaning of one to ordinary skill in medical and surgical arts.

As used herein the phrase “reduced ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “heart dysfunction” is inclusive of the ordinary and customary meaning to one of ordinary skill in medical and surgical arts, for example, heart failure or congestive heart failure.

As used herein the phrase “pericardial transection device” is inclusive of a device with a cutting surface, for example an edge of a blade or a surface of an electrode.

As used herein the phrase “pericardial incision assembly” and “incision assembly” are used interchangeable and refer to an assemblage that includes a pericardial transection device.

As used herein the phrase “transcatheter device” is inclusive of a catheter configured with at least one lumen comprising a medical instrument, device, or component thereof, for example, a pericardial transection device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein, “delta pulmonary capillary wedge pressure, A (PCWP)” is the difference between pulmonary capillary wedge pressure (PCWP) measured on volume challenge (i.e. leg raise and/or fluid administration) and PCWP at rest.

As used herein the phrase “puncturing tip” is inclusive of an atraumatic object suitable for puncturing or penetrating tissue without substantial trauma to or bleeding from the vicinity of the picture or penetration.

The present disclosure relates to an anchoring device for use with soft tissue, e.g., a pericardial layer, fibrous layer, and/or adipose tissue, as examples. Methods of anchoring a medical device, such as a pericardial transection device in soft tissue are disclosed, such as for example, a guidewire or cannula with an anchor adjacent its distal end for anchoring an end of the guidewire cannula to soft tissue, where the guidewire is subsequently used as part of a medical procedure, such as pericardial transection, whereby the guidewire or cannula, once anchored, provides a stable guide for a medical device coupled thereto. Further, as the distal end of the guidewire or cannula is anchored, the positioning of the body of the guidewire or cannula can be manipulated by, for example, pushing or pulling the guidewire or cannula in a longitudinal direction of the guidewire or cannula, with pushing of the guidewire causing bending and lateral movement of the guidewire or cannula and pulling facilitating straightening of the guidewire or cannula.

As mentioned, one example is to use the disclosed anchoring device in combination with a pericardial transection device. In this exemplary use, the distal end of the elongated body is anchored at a distal location to the pericardium. As an optional or further procedure, a reverse or backward longitudinal force directed away from the anchor device can be applied to the elongated body once anchored, which due to its distal end being anchored, causes the elongated body to straighten. The pericardial transection device can then be coupled to the elongated body, such that it can be slid along the elongated body to a selected location, with the anchor device resisting displacement of the distal end of the elongated body due to interaction with the pericardial transection device. With the transection device at a desired location and a backward tension on the elongated body, a blade from the transection device can be deployed and the transection device drawn backwards away from the distal end of the elongated body and along the elongated body to create a controlled cut path.

In an option or further embodiment, the elongated body may have a selected stiffness to allow the elongated body to be bent, flexed, twisted and/or rotated in a lateral direction relative to the longitudinal extension of the elongated body. This, in turn, will allow the elongated body to be placed at a selected location and will also cause a medical device coupled to the elongated body, such as a pericardial transection device, to also be displaced laterally to thereby control the location of the pericardial transection device. Once in a desired position, the pericardial transection procedure can be performed using the anchoring device to stabilize and control the cutting path. Once complete, the distal end of the elongated body can be decoupled from the soft tissue, either by disengaging the anchor device from the soft tissue or decoupling the distal end of the elongated body from the anchor device, to thereby remove the elongated body.

The stiffness of the elongated body can be selected based on the intended use of the device. For pericardial transection implementations, an elongated body of sufficient stiffness is desired so as to place the elongated body in the pericardial cavity and then manipulate it via force to alter it by flexing, bending, twisting, and/or rotating it so as to place a portion of the guidewire located in the body adjacent the distal end into a desired location. Stiffness can refer to a radial stiffness of the elongated body, where radial stiffness is defined as the force (newtons [N]) needed to deform the elongated body on an electromechanical dynamometer. In some embodiments, peak deformation forces (PDFs) from straight configuration to mid-wire deformation at 15 mm can be in the range of 5 to 10 N as an example. As one example, the elongated body is an extra stiff Lunderquist® guidewire with 0.035 inch Nitinol core with a PDF of 7.83±0.23 N. Other example guidewires are an Amplatz Super Stiff guidewire with PDF of 9.87±0.92 N or a Radifocus Stiff M guidewire with PDF of 7.84±0.52 N.

In some embodiments, the elongated body is a steerable guidewire or cannula, such that placement of the elongated body and the medical device coupled thereto is dictated by steering of the elongated body after it has been anchored.

With further detail of possible usage, pericardium-cutting tools or “pericardial transection devices,” depending on the configuration, can be operated during retraction—e.g. pull-to-cut or reverse cutting, or during forward cutting. This can be achieved by (i) advancing a guidewire through an entry hole in the pericardium and into the pericardial cavity, along the pericardial cavity, and to a selected location and in some instances, out of an exit hole in the pericardium, (ii) advancing the cutting tool along the guidewire, and (iii) operating the cutting tool while pulling the tool proximally.

It is possible that during step ii, the tool (which is typically more rigid than the guidewire) will draw the end of the guidewire back though the exit hole and into the pericardial cavity, thereby losing guidance. Furthermore, it would be helpful to be able to manipulate the pathway that the guidewire takes between the exit and entry holes—e.g. to form a curve that includes a particular/desirable site.

Thus, in one example, a temporary anchor (e.g. an expandable element such as a balloon, frame, or braid) at the distal end of a guidewire is provided to prevent the end of the guidewire being inadvertently drawn back into and/or through the pericardial cavity. In one example, the guidewire can be provided in a microcatheter or transcatheter device that can control the temporary anchor. The anchor can also be used as a deflection point for the guidewire, whereby through longitudinal forces to the guidewire, the guidewire can be deflected laterally to position the body guidewire and any medical device coupled thereto at a lateral position for a medical procedure.

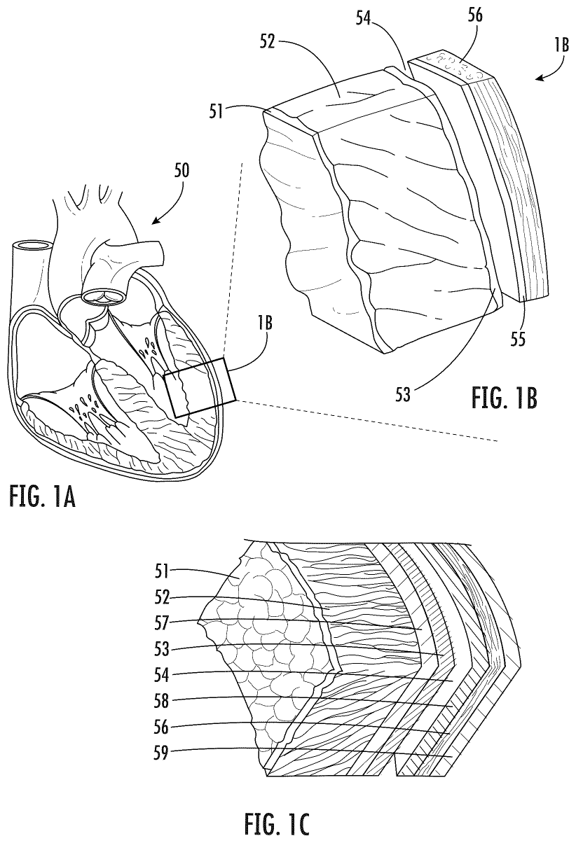

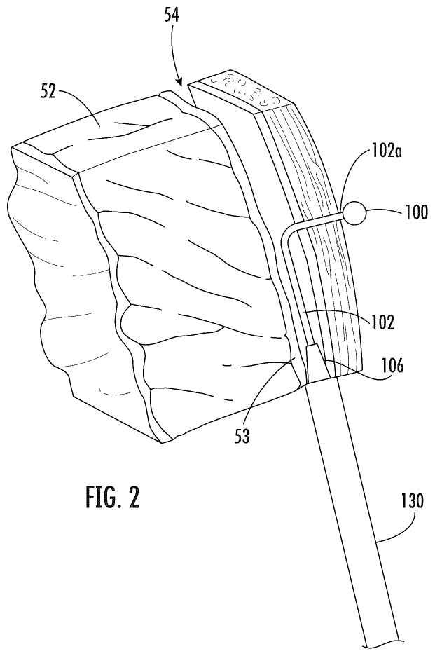

As mentioned, the anchor device of the present invention can be used as a soft tissue anchoring system. One example is pericardial tissue. With reference to FIGS. 1A, 1B, 1C, and section 1B, layers of a heart wall of a heart 50, from inside-out are depicted, being the endocardium 51, the myocardium 52, epicardial adipose tissue 57 (sometimes referred to herein as the epicardium), the visceral layer of the serous pericardium 53, the pericardial cavity 54, the parietal layer 55 of the serous pericardium 58, and the fibrous pericardium 56, and pericardial adipose tissue 59 are depicted. In one example, the presently disclosed devices are configured for introduction to the pericardial cavity 54 and for cutting tissue layers generally disposed adjacent to and including adipose tissue within and outside the pericardial cavity and superficial to the serous pericardium 53 layer of the pericardium. As a general description, the distal end of a guidewire or cannula would be introduced into the pericardial cavity 54 and advanced to a selected location, whereby an associated anchor would be deployed so as engage with the pericardium, such as the parietal layer 55 of the serous pericardium 58, fibrous pericardium 56, and/or the pericardial adipose tissue 59.

For anchoring the distal end of the guidewire. In some instances, the distal tip of the guidewire can be used to puncture through these layers and out of the pericardium so that the anchor is deployed above the pericardial adipose tissue 59 layer. Once the anchored is deployed, a medical device, such as a pericardial transection device can be advanced and/or retracted along the guidewire for performing cuts in the pericardial layers. In some embodiments, positioning of the pericardial transection device can be controlled by manipulating deflection of the guidewire body relative to the anchor so as to place the transection device at different lateral locations in the pericardial cavity for performing cutting operations. In some embodiments, the guidewire can be deflected to different locations so as to create multiple cuts at different locations in the pericardial tissue, using the one anchoring point.

With reference to FIG. 2, an exemplary implementation of the disclosed anchoring device is illustrated. As shown, an anchoring device comprising an anchor device 100 is connected at or near the distal end 102a of an elongated body 102 in the form of a guidewire or cannula. The guidewire 102′ in this embodiment is housed in a catheter 104 and extending from an insertion tip 106. The distal end of the guidewire has been introduced and guided through the pericardial cavity 54 and has punctured through the parietal layer 55 of the serous pericardium 58, fibrous pericardium 56, and/or the pericardial adipose 59 layers to a position external of the pericardium at a distal location at a selected position 140. In this configuration, the guidewire represents an elongated body for use in a medical procedure comprising a proximal end, not shown, and a distal end 102a. The anchor device 100 is coupled adjacent to the distal end 102a of the guidewire for anchoring the elongated body to soft tissue, which in this case is the outer surface of the pericardium. The anchor device 100 has first and second configurations. In the first configuration, the anchor device is in a non-deployed state so as to be configured to avoid attachment or otherwise not secure itself to the soft tissue, and in a second configuration, the anchor device is in a deployed state so as to secure itself to the soft tissue.

As illustrated in FIG. 2, in one embodiment, the anchor device 100 comprises an expandable body configured for transitioning from the first configuration in a compressed state to an expanded state in the second configuration to thereby press the anchor against the soft tissue. In one embodiment, in the compressed or deflated state, the expandable body has a first outer diameter for traversing and location in a catheter or in a body cavity, such as a pericardial cavity, and a second outer diameter larger than the first outer diameter when in expanded state for coupling to soft tissue. For example, the anchor device 100 can be an inflatable bladder, such that in the second configuration, the bladder is inflated to thereby press the anchor against the soft tissue. The bladder, when inflated, is larger than the hole punctured into the pericardial tissue by the distal end of the guidewire 102′, such that the distal end of the guidewire is “anchored” relative to the pericardium. As the anchor device 100 is expandable and contractible, for example, by use of a remote pump, not shown, in fluid communication with the anchor device, the anchor device can be expanded to provide an anchoring function in a second configuration and retracted to disengage from the soft tissue in a first configuration.

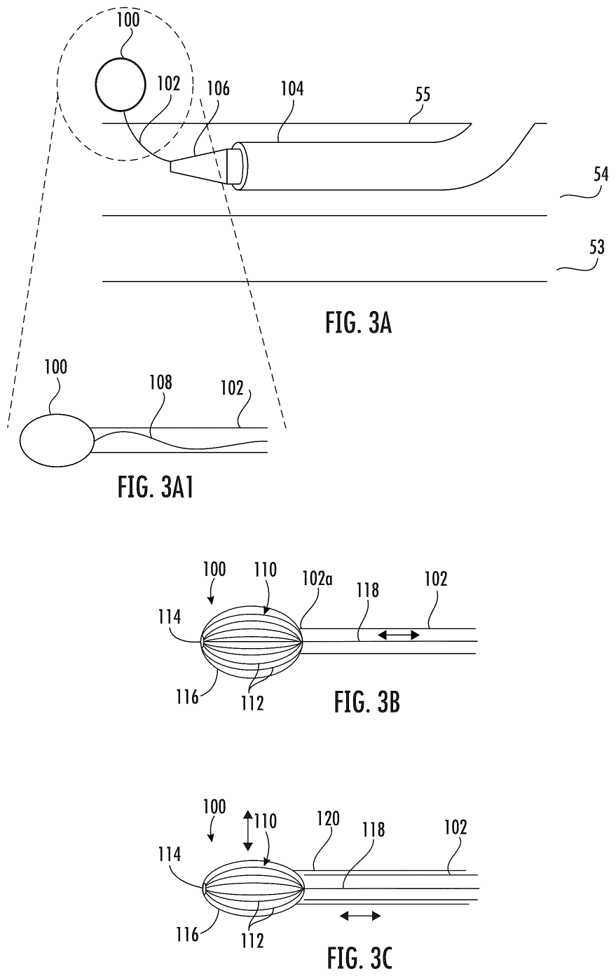

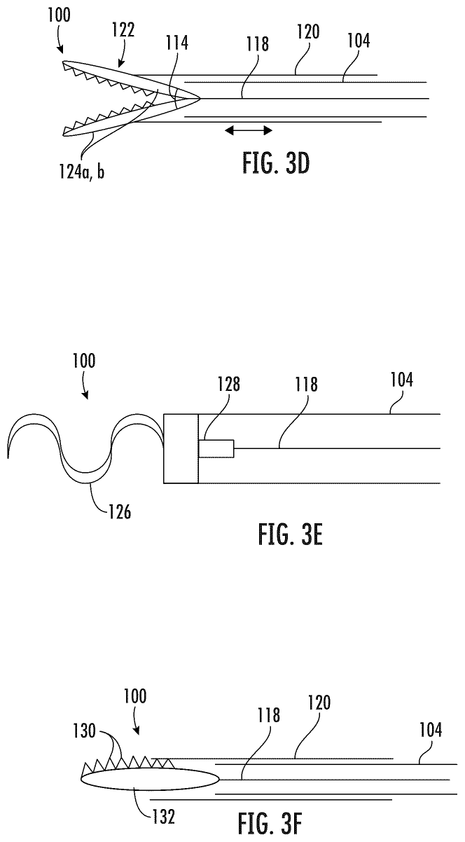

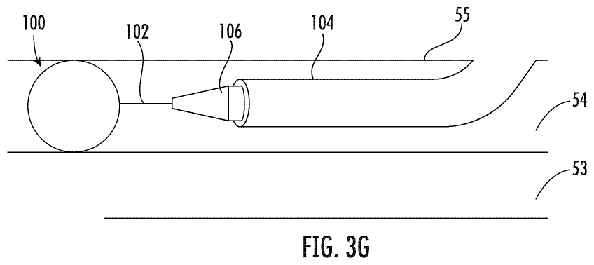

As illustrated in FIGS. 3A & 3A1, in one embodiment, the guidewire 102′ is a tube structure with a central bore (i.e., lumen) 102c therethrough. For example, the guidewire could be a cannula with a sufficiently stiff/rigid body for navigating within the pericardial cavity and a central bore. The anchor device 100 of this embodiment, is an inflatable bladder with an inlet opening connected to a tube 108 located in the central bore 102c of the guidewire 102′. The tube in the central bore 102c is in fluid communication with a remote pump, which is operable to either supply positive air or a vacuum to the anchor device 100 to thereby inflate and deflate the device. For example, in one embodiment, the anchor device and guidewire can be configured so as to be deployed in a multi-lumen catheter, where the guidewire is located in one lumen and the anchor is deployed from another lumen, where the lumen or a tube in the lumen supplies fluid to the anchor device to inflate or deflate the anchor. In embodiments where a separate lumen or tube are provided to control inflation and deflation of the anchor, the guidewire could be a solid body or wire with no central bore.

With reference to FIG. 3B, in one or further embodiments, the anchoring device includes an inner frame 110 that is expandable and contractable. For example, the inner frame 110, can be a series of parallel arms or struts 112 cut from a tube, such as a rigid plastic or metal tube, where the distal ends of the struts 112 are coupled or form part of a distal hub 114. A control wire 118 is connected to the distal hub and fed through a central bore formed by the inner surfaces of the struts. The arms/struts are collapsible and radially expandible, such that when expanded in a second configuration they contact soft tissue for anchoring and in a collapsed configuration can avoid contact with soft tissue. As depicted in FIG. 3B, the inner frame 110 can be positioned inside an inflatable bladder 116. When the struts are forced apart, such as by a longitudinal force applied to control wire 118 which pulls the distal hub 114 toward the distal end 102a of the lumen, which captures the proximal ends of the struts and causes the struts to be compressed outwardly thereby stretching the bladder outwardly, the anchor is expanded to engage the soft tissue in the second configuration. When the control wire 118 is relaxed or pushed backwards, the struts 112 of the inner frame 110 collapse toward each other, thereby causing the bladder to retract placing the anchor device in a non-deployed first configuration not in contact with the soft tissue.

With reference to FIG. 3C, in one or further embodiments, the struts 112 are outwardly biased. In this embodiment, a sheath 120 defining a lumen therein is positioned so as to be movable longitudinally relative to the lengthwise extension of the struts so as to be operably slid over the struts to compress them to the first configuration and to slide away from the struts, allowing the struts to expand outwardly in the second configuration so as deploy as an anchor.

With reference to FIG. 3D, in one or further embodiment, the anchor device can be configured as a clamp 122 comprising two opposed arms 124a, 124b, biased outwardly by a spring 128 or other bias mechanism. In one embodiment, a sheath 120 forming a lumen is positioned so as to be movable longitudinally relative to the opposing arms so as to be slid over the arms to compress them to the second configuration and to slide away from the arms, allowing the arms to expand outwardly in the first configuration. In the second configuration, the arms can be brought together by entering the lumen in the sheath to clamp onto the soft tissue and in the first configuration release the soft tissue. As opposed to a slidable sheath, in one embodiment, the movement of the opposed arms can be actuated by a wire that, when pulled, brings the arms together in the second configuration and when pushed or released, allows the arms to separate to the first configuration.

With reference to FIG. 3E, in one or further embodiment, the anchor device is a helical screw 126. In this embodiment, the helical screw is connected at or adjacent to the distal end of the guidewire. In one embodiment, to engage the anchor with soft tissue, the guidewire can be twisted so as to rotate the anchor thereby driving the helical screw into the tissue. In an alternative embodiment, a torsion spring 128 can be located between the proximal end of the anchor and the distal end of the guidewire. The spring could be wound prior to deployment. To engage the anchor with the soft tissue, the tension on the torsion spring could be released, causing the torsion spring to rotate and thereby rotate the helical screw of the anchor device into the soft tissue.

Regarding the embodiment of FIG. 3E, disengaging the distal end of the guideline from the soft tissue following a medical procedure can be accomplished in several ways. For example, in one embodiment, the guidewire could be twisted/rotated counter-clockwise to unscrew the anchor. In some embodiments, the anchor can be disengaged from the distal end of the guidewire and left attached to the soft tissue. This could be achieved by use of a weakened break away section on the proximal end of the anchor that can be used to separate the distal end of the guidewire from the anchor. In some embodiments, there can be a connector connecting the distal end of the guidewire to the proximal end of the anchor that can be disconnected. In these embodiments, the anchor can be left in the soft tissue. In some embodiments, the anchor is formed of a dissolvable material, allowing the anchor to dissolve away, once no longer in use.

With reference to FIG. 3F, in one or further embodiment, the anchor device 100 comprises one or more protruding barbs 130 configured for attaching to the soft tissue. In one embodiment, the anchor device comprises a central body 132 extending between distal and proximal ends with one or more protruding barbs located on the central body. In one embodiment, the protruding barbs are fixed to the central body and in an alternative or further embodiment, the one or more barbs are retractable and deployable whereby they movable laterally relative to the central body and biased outwardly by a spring or other biasing mechanism. Associated with the anchor device is a movable sheath 120 forming an internal lumen that can be slid over the protruding barbs. When the movable sheath is located over the protruding barbs in a second configuration (i.e., the barbs have been slid into the lumen in the sheath), the protruding barbs are prevented from contacting and anchoring to any soft tissue. When the anchor device is to be deployed so as to attach to the soft tissue in a first configuration, the sheath can be slide relative to the central body in a longitudinal direction so as to place the protruding barbs in a position to attach to the soft tissue. Removal of the anchor device, following use, can be achieved by extending the sheath to cover the protruding barbs.

In yet another or further embodiment, the protruding barbs are operable connected to a control wire that can be manipulated longitudinally relative to the central body to either extend or retract the protruding barbs from the central body to place the barbs selectively in the first or second configuration.

With reference to FIG. 3G, in one or further embodiment, the anchor device 100 comprises an expandable anchor device 100, such as an inflatable or expandable bladder. In this embodiment, the anchor device is deployed in the pericardial cavity 54. The anchor device 100 in the second configuration is expanded so as to press against both the parietal layer 55 of the serous pericardium 58 and the visceral layer of the serous pericardium 53 with sufficient force to anchor the distal end of the elongated body 102 relative to the pericardium. In some embodiments, the outer surface of the bladder of the anchor device can include bumps, grips, or roughened areas to provide increased friction between the bladder and the layers of the pericardium to increase anchor strength.

As discussed above, in one or more embodiments, the anchor device is connected to an elongated body in the form of a guidewire and the anchor is connected adjacent to or at the distal end of the guidewire. In some embodiments, the anchor device is connected to an elongated body in the form of a catheter, whereby the anchor is connected adjacent to at the distal end of the catheter.

In some embodiments, the elongated body is a multi-lumen catheter or a microcatheter. In some embodiments, the anchor device comprises a guidewire having an anchor adjacent to or at the distal end of the guidewire. The guidewire and anchor are configured for placement in a catheter for deployment, such that when positioned in the body and navigated to an engagement location, the anchor is positioned in the catheter so as to avoid contact with soft tissue. Once at the engagement location, the anchor can be extended from the catheter to engage with the soft tissue. Once anchoring is no longer required, the anchor can then be retracted back into the catheter for removal from the body.

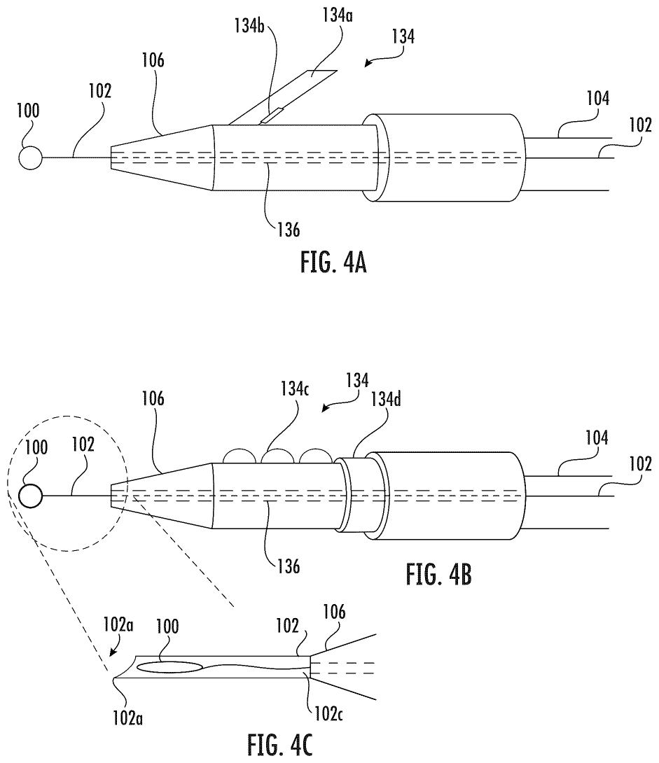

With reference to FIG. 4A, in connection with the anchor device 100, the elongated body can have a medical device, such as a pericardial transection device 134 coupled thereto. FIG. 4A depicts a transection device 134 in the form of cutting knife 134a having an electrified section 134b. The transection device is coupled to a guidewire or cannula 102″, such as via a collar or central bore (i.e., lumen) 136 associated with the medical device for slidably connecting the medical device to the guidewire or cannula 102″. For example, in some embodiments, the elongated body is a guidewire or cannula, with a transection device 134 coupled to the body via a collar 136 or other similar connection. The transection device could be a scalpel, a mechanical cutting device, an electrode, an RF electrode, an electrosurgical device, a reversibly retractable knife blade, or combination of any two or more thereof. In some embodiments, a probe is connected to the elongated body configure for nerve stimulation or detection. In some embodiments, the elongated body and/or the anchor includes a portion thereof comprising radiopaque material. Further, as will be understood, the anchor, elongated body, and/or any medical device coupled therewith are, in some embodiments, sterilized and made of sterilizable materials.

FIG. 4B discloses an alternative embodiment where the transection device 134 is an electrode or RF electrode device with a series of conductive wires 134c that when electrified act to cut tissue. A slidable sheath 134d defining an inner lumen is located in relation to the series of wires 134c and the catheter so as to selectively expose the series of wires when in position to perform a cutting operation. Located in the transection device is a lumen, collar 136 or through hole sized to receive the guidewire 102′ or cannula extending through the through hole. Here the elongated body forming the guidewire or cannula includes a central bore (i.e., lumen), not shown, with a tube located therein that is in fluid communication with the anchor device 100. In this depicted embodiment, the anchor device 100 is an inflatable balloon for receiving fluid/air from the tube for inflating and deflating the balloon of the anchor device.

As illustrated in FIG. 4C, the distal end 102a of the elongated body 102 can include a pointed or piercing section for use in piercing soft tissue, such as the pericardium. In a non-deployed first configuration, the anchor device 100, such as an inflatable balloon can be located in the central bore 102c of the elongated body near the distal end. In this configuration, the distal end 102a of the elongated body, via the pointed or piercing section, can be used to position the distal end of the elongated body at a desired location to deploy the anchor device 100. After proper location, the anchor device 100 can then be deployed in a second configuration to engage with the soft tissue.

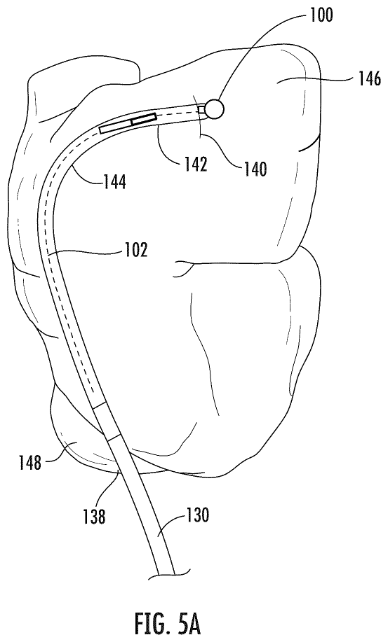

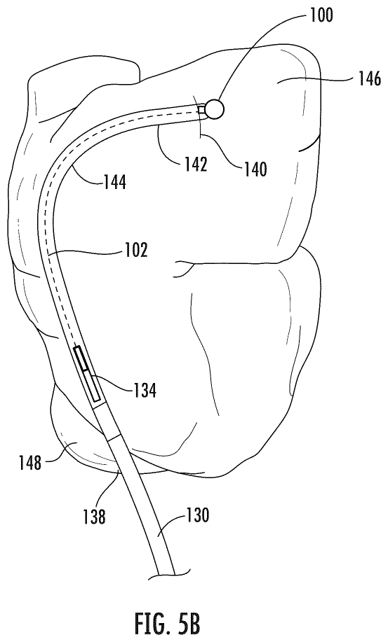

With reference to FIGS. 2, 5A, and 5B, deployment and use of an anchor device 100 according to one embodiment of the present invention is further described. With reference to FIG. 5B, a transection device 134 is shown where a flexible catheter 104, a guidewire or cannula 102″, and an anchor device 100 are advanced through a punctured entry hole or access point 138 through the pericardium and into the pericardial cavity 54, within/under the parietal layer 55 of the serous pericardium 58, and the fibrous pericardium 56, and pericardial adipose tissue 59 and advanced to a cut-start site or target site 142 (which is at the distal end of the path to be cut) in its undeployed configuration. As shown, in one example, the distal end of guidewire 102′ and the anchor device 100 are caused to exit the pericardial cavity 54. The anchor device is then deployed by inflation or expansion to thereby anchor the distal end of the guidewire 102′ relative to the target site 142 at an anchor spot.

In this configuration, an anchored medical device is provided. The anchored medical device comprises an elongated body in the form of a guidewire 102′ spanning between a proximal end and a distal end. An anchor device 100 coupled adjacent to the distal end for anchoring the guidewire 102′ to the soft tissue, the anchor device having first and second configurations, wherein in the first configuration the anchor is configured to avoid attachment to or otherwise not secure itself to soft tissue, and in the second configuration the anchor is configured to secure itself relative to at least a portion of the soft tissue, wherein a portion of the elongated body in the pericardial cavity or a pericardium and the anchor device is in the second configuration and is secured to a portion of the pericardium. The transection device 134 is located in the pericardial cavity and in contact with at least the parietal layer 55 of the serous pericardium layer 58 of the pericardium.

As illustrated in FIG. 5B, once the anchor device is inflated in anchoring position, a cutting surface or RF electrode of the transection device 134 is then extended and the transection device 134 is retracted along a cutting path 144—i.e. the cutting is performed while pulling the device back toward the punctured entry hole, for example, along guidewire 102′ with transection device 134 in its deployed configuration. Thus, pre-cutting advancement of the device is employed by advancing the anchor and distal end of the guidewire out of the pericardium at the cut-start target site 142 and then, once anchored, pulling the cutting device or assembly backwards along the cut path.

In the illustrated depiction, the at least one incision length is a path 144 made in the pericardium from an anterior apex 146 to posterior base 148 of a heart using a pericardial transection device 134 configured for reverse cutting (i.e. going over the middle cardiac vein and terminating the incision at the inferior vena cava). In one example, the at least one incision length is a path from an apex of a heart to a posterior right atrium and terminating at the superior vena cava. In one example, the at least one incision length is a path from an apex of a heart to a coronary sinus. In one example, the at least one incision length is a path from an apex of a heart to an ascending aorta. In one example, the at least one incision length is a path transversely about a heart (i.e. from a right ventricle to a left atrial appendage). The incision lengths, in a heart with a dysfunction treatable with the present method, cause the pericardium to separate radially about the cut line, without the removal of pericardial tissue. Other incision lengths and paths can be employed, as well as combinations of incision lengths and paths, and combination of incision lengths and paths with one or more of partial removal of pericardium, drainage, and other pericardial treatments.

In one example, pairs or bars of radiopaque markers on opposite sides of the device are positioned on the pericardium or within the pericardial cavity along the target cut path while the device is being advanced in the pericardial cavity to the starting point of the cut. As the anchored transection device is operated, a fluoroscope or other imaging technique can be used to visualize the separation of the pairs or bars of radiopaque markers, thereby indicating the totality of the at least one incision.

In one example, the at least one incision length and path is preoperatively determined. In another example, creating at least one incision length is determined in response to a signal indicative of a reduction of restraint of the heart. In one example, creating at least one incision length is determined in response to a signal indicative of a reduction of restraint of the heart. In one example, creating at least one incision length is determined in response to a signal indicative of a reduction of restraint of the heart; and repeating the creating of at least one incision length. In one example, the presently disclosed method further comprises, after creating the at least one incision length, confirming a location of a distal end of the pericardial transection device; and in response to a signal indicative of a reduction of restraint of the heart, repeating the steps of creating the at least one incision length, and confirming a location of the distal end.

The pericardial transection device that can be used with the presently disclosed anchor device can be any soft tissue cutting device, using a blade, current carrying wire, or combination thereof. The pericardial transection device can be a “scissor-like” instrument with more than one sharp edges. The pericardial transection device can be stabilized with one or more support members, such as wires or inflatables or balloons to assist the presently disclosed anchor device during anchoring with the pericardium/parietal layer. The support members can be passively or actively deployed during the procedure. The pericardial transection device that can be used with the presently disclosed anchor device can be configured to bulge, stretch, or otherwise tension the pericardial space before or during anchoring.

Exemplary pericardial transection devices that can be used with the presently disclosed anchor device are not limited, and can include, for example, soft tissue cutting devices and the like. Exemplary pericardial transection devices can also include, for example, electrocautery devices.

The pericardial transection device and/or catheter and/or sheath can be configured such that the total outer diameter (O.D.) introduced to the pericardial cavity is between about 6 Fr (2 mm) and about 30 Fr (10 mm). The pericardial transection device and/or catheter and/or sheath can be configured such that the total outer diameter (O.D.) introduced to the pericardial cavity is between about 6 Fr (2 mm) and about 20 Fr (6.67 mm). The pericardial transection device and/or catheter and/or sheath can be configured such that the total outer diameter (O.D.) introduced to the pericardial cavity is between about 6 Fr (2 mm) and about 15 Fr (5 mm). The pericardial transection device and/or catheter and/or sheath can be configured such that the total outer diameter (O.D.) introduced to the pericardial cavity is between about 6 Fr (2 mm) and about 12 Fr (4 mm). The pericardial transection device and/or catheter and/or sheath can be configured such that the total outer diameter (O.D.) introduced to the pericardial cavity is approximately 10 Fr (3.33 mm).

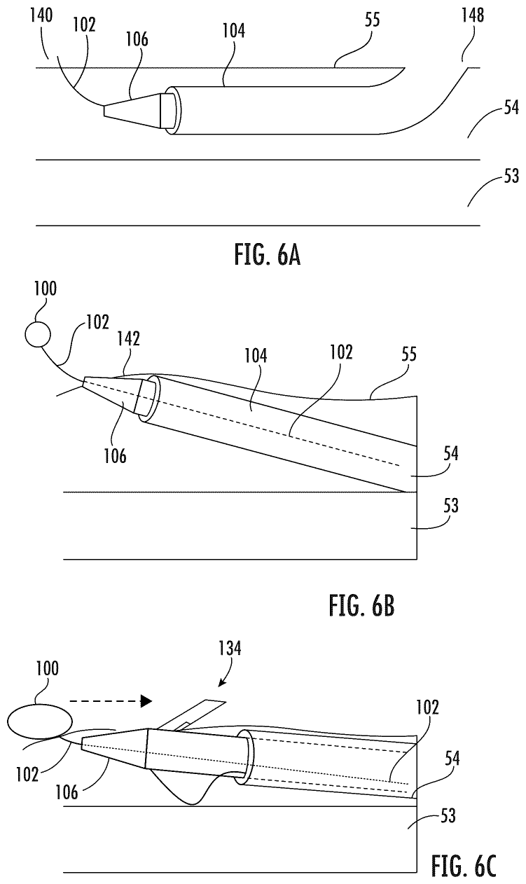

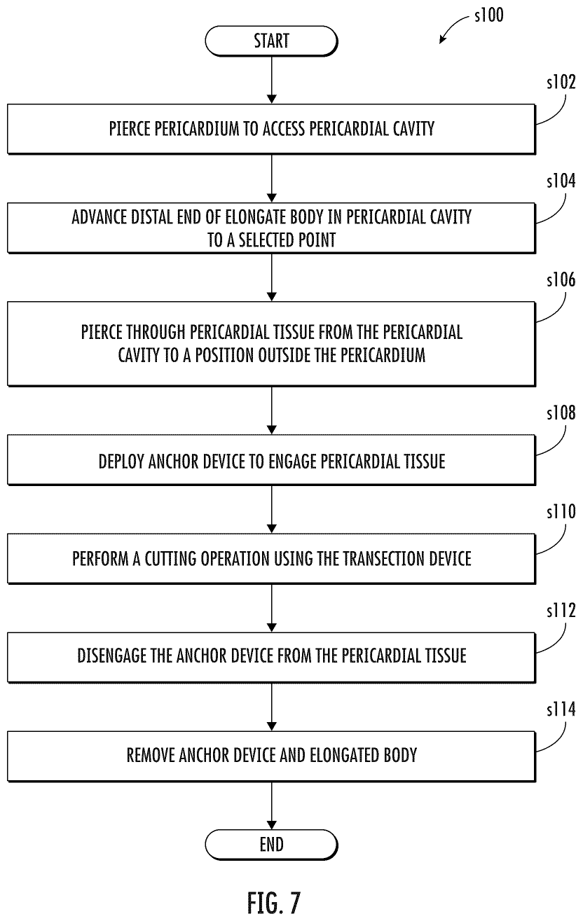

With reference to FIGS. 6A-6C And FIG. 7, a series of operations are depicted to deploy and use an anchor device 100 in conjunction with a transection device 134. Specifically, as illustrated in FIG. 6A and FIG. 7, in operation (s100), the pointed or piercing section of the distal end of the elongated body 102 (here a guidewire or cannula with central through bore) is used to pierce a subject pericardium (s102) and then the distal end of the elongated body 102 is advanced along the pericardial cavity 54 to a selected position 140 (s104).

With reference to FIG. 6B, the depicted embodiment illustrates an embodiment where the anchor device 100 is deployed to anchor at a position on an external surface of the pericardium. In this embodiment, the distal end of the elongated body 102 is advanced via steering of the elongated body, so that the pointed or piercing section punctures through the parietal layer 55 of the serous pericardium 58, and the fibrous pericardium 56, and pericardial adipose tissue 59 and out of the pericardium (s106).

At this position, the anchor device 100 is inflated in a second configuration so as to have a dimension, such as a diameter, that is larger than the hole created by the distal end of the guidewire (s108). In this configuration, the distal end of the elongated body is thereby “anchored” to the soft tissue in a rigid or partially rigid manner.

With reference to FIG. 6C, following deployment of the anchor device 100 in the second configuration, the transection device 134 deployed on the elongated body 102 can now be used to create cuts in the pericardium by pulling backwards away from the distal end of the elongated body the transection device with the cutting portion engaging the pericardial tissue (s110). In this configuration, the anchor device 100 provides stability and accuracy to the cutting actions of the transection device 134 by creating a rigid and defined cut path via the elongated body.

Once the transection device has been operated to create one or more cuts in the pericardial tissue, the elongated body can then be disengaged from the pericardial tissue by deflating the anchor device 100 back to a first configuration whereby the anchor device is no longer rigidly or semi-rigidly attached to the pericardial tissue (s112). This disengagement of the anchor device from the pericardium, then allows the clinician to draw backwards the elongated body 102 to thereby remove it from the pericardium and eventually from the patient's body (s114).

The above embodiment envisions placement of the anchor at an exterior position of the pericardium. The procedure regarding placement of the anchor device depends, somewhat, on the type of anchor used. If the anchor device is a helical screw or a clamp or similar “grasping type” device, then the anchor device would be deployed to engage with the parietal layer 55 of the serous pericardium 58 and possibly portions of the fibrous pericardium 56 above the pericardial cavity 54. In some embodiments, where the anchor device is an inflatable balloon, the balloon could be sized sufficiently such that once inflated, it expands to engage the wall of the pericardial cavity 54 to thereby anchor the distal end of the elongated body 102 within the pericardial cavity 54. While the above example describes an instance where they anchor device is an inflatable balloon, it will be understood that any of the other configurations of the anchor device described herein can be used in a similar manner as described above. Some of the anchor device configurations may not require a second puncture in the pericardium so as to place the anchor device on an exterior surface of the pericardium. For example, if a helical screw or a clamp is employed, as opposed to an inflatable balloon, then the anchor device can be deployed in a second configuration to anchor to the pericardium while the anchor device remains in the pericardial cavity.

One optional benefit of the use of an anchor device is the ability to use the anchor device, once deployed in the second configuration, to selectively place the transection device 134 relative to the pericardium for the cutting procedure. Flexibility and bend characteristics of the elongated body may be used to control the positioning of the elongated body for use in a subsequent medical procedure, such as pericardial transection. For pericardial transection implementations, an elongated body of sufficient stiffness is desired so as to place the elongated body in the pericardial cavity and then manipulate it via force to alter it by flexing, bending, twisting, and/or rotating it so as to place a portion of the guidewire located in the body adjacent the distal end into a desired location. Stiffness can refer to a radial stiffness of the elongated body, where radial stiffness is defined as the force (newtons [N]) needed to deform the elongated body on an electromechanical dynamometer. In some embodiments, peak deformation forces (PDFs) from straight configuration to mid-wire deformation at 15 mm can be in the range of 5 to 10 N as an example. As one example, the elongated body is an extra stiff Lunderquist® guidewire with 0.035 inch Nitinol core with a PDF of 7.83±0.23 N. Other example guidewires are an Amplatz Super Stiff guidewire with PDF of 9.87±0.92 N or a Radifocus Stiff M guidewire with PDF of 7.84±0.52 N.

As an optional or further procedure, a reverse or backward longitudinal force directed away from the anchor device can be applied to the elongated body once anchored, which due to its distal end being anchored, causes the elongated body to straighten. The pericardial transection device can then be coupled to the elongated body, such that it can be slid along the elongated body to a selected location, with the anchor device resisting displacement of the distal end of the elongated body due to interaction with the pericardial transection device. With the transection device at a desired location and a backward tension on the elongated body, a blade from the transection device can be deployed and the transection device drawn backwards away from the distal end of the elongated body and along the elongated body to create a controlled cut path.

In an option or further embodiment, the elongated body may have a selected stiffness to allow the elongated body to be bent, flexed, twisted and/or rotated flex/bend in a lateral direction relative to the longitudinal extension of the elongated body. This, in turn, will allow the elongated body to be placed at a selected location and will also cause a medical device coupled to the elongated body, such as a pericardial transection device, to also be displaced laterally to thereby control the location of the pericardial transection device. Once in a desired position, the pericardial transection procedure can be performed using the anchoring device to stabilize and control the cutting path. Once complete, the distal end of the elongated body can be decoupled from the soft tissue, either by disengaging the anchor device from the soft tissue or decoupling the distal end of the elongated body from the anchor device, to thereby remove the elongated body.

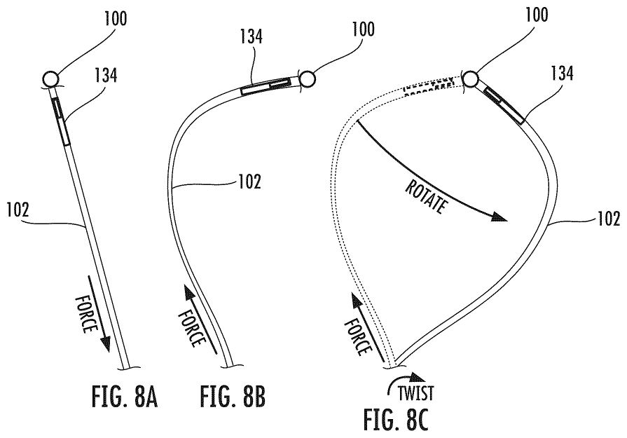

FIGS. 8A-8C are diagram-type views of example manipulations of the elongated body 102 once anchored by an anchor device 100 to soft tissue. As illustrated in FIG. 8A, once anchored, a reverse or backward longitudinal force directed away from the anchor device can be applied to the elongated body once anchored, which due to its distal end being anchored, causes the elongated body to straighten. The pericardial transection device 134 can then be coupled to the elongated body, such that it can be slid along the elongated body 102 to a selected location, with the anchor device resisting displacement of the distal end of the elongated body due to interaction with the pericardial transection device. With the transection device 134 at a desired location and a backward tension on the elongated body, a blade from the transection device can be deployed and the transection device drawn backwards away from the distal end of the elongated body and along the elongated body to create a controlled cut path.

As illustrated in FIG. 8B, the location for the transection device 134 and its cut path can be altered by applying a longitudinal force in a direction toward the anchor device. This, in turn, causes the elongated body to bend or curve into a different position. As illustrated in FIG. 8B, the cut path for the transection device 134 will now follow the curve of the elongated body 102 to create a curved transection cut.

As illustrate in FIG. 8C, the elongated body 102 may be manipulated by applying both a longitudinal force in a direction toward the anchor device and also twisting or rotating the elongated body. This causes the elongated body 102 to orient into yet a third position, such that a third cut path may be realized with the transection device 134.

As will be understood any number of directional and/or combinations of forces can be applied to the elongated body, once anchored, to manipulate the shape of the elongated body so as to place the transection device in a desired location and so as to create a desired cut path.

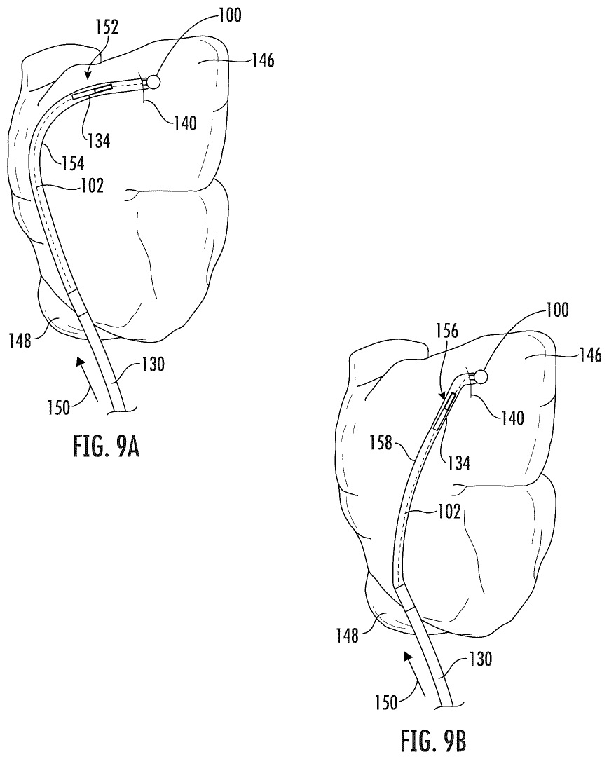

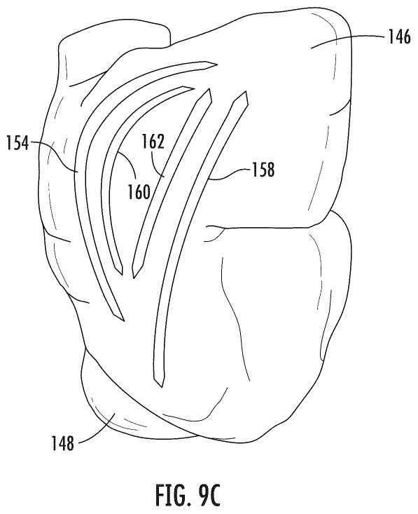

For example, FIGS. 9A-9C depict a series of views whereby the elongated body 102 in an anchored position, can be used for placement of the transection device at different locations in the pericardial cavity for making one or more transections.

FIG. 9A illustrates an initial position of the anchor device 100, elongated body 102, and the transection device 134, with the anchor device in a second configuration, whereby the anchor device is engaged with the pericardium, and the transection device 134 and elongated body 102 located in the pericardial cavity 54. In the illustrated embodiment, the elongated body is a guidewire or cannula with a degree of flexibility that allows the elongated body to bend when a longitudinal force is applied thereto, whereby, due to the force of the anchor device, the elongated body is caused to flex of deflect laterally.

Specifically, with reference to FIG. 9A, a longitudinal force 150 has been applied to the elongated body 102. Due to this force 150, the action of the anchor device, and the flexibility of the elongated body or at least a portion of the elongated body, the elongated body and the transection device 134 coupled thereto are displaced laterally to a new position 152. From this new position 152, the transection device 134 could be deployed to make a first line of cut 154 in the pericardium.

With reference to FIG. 9B, a longitudinal force 150 has been applied to the elongated body 102. Due to this force 150, the action of the anchor device, and the flexibility of the elongated body or at least a portion of the elongated body, the elongated body and the transection device 134 coupled thereto are displaced laterally to a new position 156. From this new position 156, the transection device 134 could be deployed to make a first line of cut 158 in the pericardium.

As illustrated in FIG. 9C, several different cut lines or paths 154, 158, 160, 162 can be developed in the pericardium by applying different levels of force and/or directions of force 150 on the elongated body 102. For example, the transection device 134 has been placed at various starting locations via manipulation of force on the elongated body 102 to thereby create a series of cuts in the pericardium at the laterally displaced location.

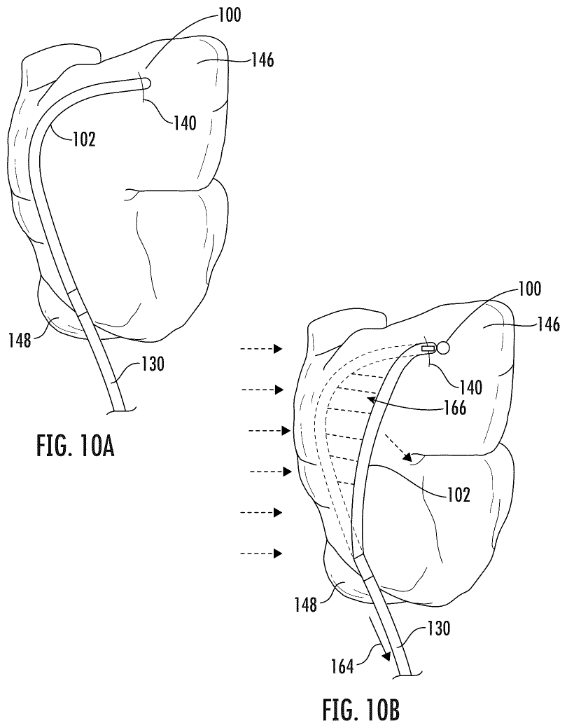

With reference to FIGS. 10A-10B, in some embodiments, both tension 164 on the elongated body 102 and a pulling or pushing force on the transection device 134 could be used to create two or more spaced apart cuts in the pericardium at the same time. For example, as illustrated in FIG. 4b, the transection device 134 could have a series of cutting blades or electrical wires 134c that are spaced apart along the pericardial transection device 134. In this configuration, both the elongated body could be pulled in a longitudinal direction 164 and the pericardial transection device 134 pulled in a longitudinal direction to thereby create cuts 166 in a trans-longitudinal or trans-lateral direction.

As discussed, relative to FIGS. 8A-8C, 9A-9C, and 10A-10B, the location of the elongated (e.g., guidewire or cannula) in the pericardial cavity and hence a medical device (e.g., pericardial transection device) can be affected by forces applied to the elongated body to cause it to flex and move within the pericardial cavity. As will be understood other forces could be applied individually or in combination with other forces. For example, in one embodiment, the location of the elongated body in the pericardial cavity can be manipulated via rotation of the elongated body or by applying a torsional force thereto. In another embodiment, both applying a rotation and a longitudinal force to the elongated body will cause it to change location in the pericardial cavity.

As mentioned above, the anchor device provides for creating incision totalities, using an anchored pericardial transection device, of an incision position, length, and width in at least the parietal layer, fibrous layer, or adipose layer of the pericardium. The anchored pericardial transection device with at a single cutting surface or a plurality of cutting surfaces can create an extended incision length and width, a segmented incision length, or a combination thereof. The extended incision length in one example, creates a curvilinear opening in at least the pericardium/parietal layer. The curvilinear opening can be of varying width (measured transversely) from the entry point to the anchored point.

The cutting surface can be a knife or scalpel or an electrode. In one example, the at least one incision length is a path along a circumference of the pericardium of a length sufficient to reduce pericardial restraint. The incision length can be made, either subxiphoidally, or transvascularly, by advancing a pericardial transection device, from the initial pericardial cavity entry location with cutting to a distal location (forward cutting). Alternatively, the incision length can be made by traversing the pericardial transection device from the initial pericardial cavity entry location to a distal location and then reversing back towards the entry location with cutting (reverse cutting). Thus, the presently disclosed method can be used with pericardial transection devices that are configured for either forward or reverse cutting modalities.

The embodiments described in FIGS. 8A-8C, 9A-9C, and 10A-10B describe an elongated body in the form of a guidewire or cannula having a degree of flexibility such that forces applied to the elongated body cause it to flex and/or rotate to change positions of the elongated body in the pericardial cavity. In one or further embodiments, the elongated body is a steerable guidewire or cannula, such as known in the art. For example, the elongated body, in one embodiment, comprises segments body segments connected by shape memory alloy that can be used in conjunction with guidewire(s) to steer the body segments.

The presently disclosed anchor device can be used when entry/exit sites are provided through the subxiphoid region of the patient (subxiphoid approach). The presently disclosed anchor device can be used when entry/exit sites are provided transvascularly.

As will be understood a steerable catheter can be employed having a plurality of steerable segments. In some examples, the steerable catheter guiding the pericardial transection device 134 can have a radius of curvature of between about 1 inch and about 5 inches, with an arc length of between about 90° and about 180°. As discussed, the pericardial transection device is capable of cutting one or more layers of the pericardium, and in some instances, at least a portion of the parietal layer 55 of the serous pericardium 58, and the fibrous pericardium 56, and pericardial adipose tissue 59 are cut and separated. The one or more incisions along the one or more lengths, in a heart with a dysfunction treatable with the present method, cause the pericardium to separate about the length of cut paths, without the removal of pericardial tissue. With the steerable catheter, which be repositioned using the same or different anchoring points, can create various cut lengths along cut paths. One or more cut paths can be made, and different cut paths, of various lengths, e.g., segmented, continuous, or combinations, can be made in at least a portion of the parietal layer and other pericardium tissue to reduce pericardial restraint. In one example, the cut path and its length are pre-operatively determined. Other cut totalities (positions, paths, and lengths) can be used.

In one example, an ultrasound method can be used to visualize the device and/or to measure the hemodynamic parameter. In some such examples, echocardiography is used. In some examples, a contrast dye is used. In yet another example, a fiber optic is used for visualization.

In some examples, fluoroscopy is used to visualize the position of the cannula and/or the anchor device as it is advanced towards the heart. Visualization can be enhanced by incorporation of a radiopaque marker on one or more of the cannula and/or the anchor device. In one example, the cannula is visualized from the left anterior oblique view at approximately 35-40°. In one example, the user can inject a contrast solution through the cannula or through a lumen of the pericardial transection device as they are being advanced towards the heart.

Part or all of the medical device (e.g., the multi-lumen catheter, anchor device, the pericardial incision assembly, the pericardial transection device, the guidewire, and controller) can be sterilized for use. Sterilization includes aseptic sterilization. The medical device can be sterilized using various sterilizing techniques, such as E-Beam sterilization, gamma sterilization, ethylene oxide sterilization, autoclave sterilization, chemical sterilization, and/or the like. Additionally, one or more materials used in the medical device can have anti-bacterial characteristics.

In one example, the anchor device can further comprise at least one nerve detection device. In one example, the at least one nerve detection device is located on the flexible catheter 104. In one example, the at least one nerve detection device is located adjacent the pericardial transection device 134. In one example, the at least one nerve detection device is located on the insertion tip 106. In one example, the at least one nerve detection device is located on the pericardial transection device 134.

In one example, a pacing catheter or pacing electrode can be used in combination with anchor device 100. In another example, anchor device 100 can be signal guided during deployment and/or during incising/cutting of the pericardium, for example, using impedance, echocardiography, or other methods.

In one example, the pericardial transection device 134 can further comprise at least one nerve proximity device. In one example, the at least one nerve proximity device is a nerve detection device, for example, a pacing device or catheter introduced via a lumen in the transcatheter of the pericardial transection device. In one example, the at least one nerve detection device is located on a flexible catheter. In one example, the at least one nerve detection device is located adjacent or within the pericardial transection device 134. In one example, the at least one nerve detection device is located on the insertion tip 106. In one example, the at least one nerve detection device is located on the cutting surface. In one example, the at least one nerve detection device provides a proximity indication to at least a portion of a nerve, e.g., the phrenic nerve. The at least one nerve detection device can be an impedance sensor or other electrical field detection device that can provide a visual, audio and/or tactile indication to the user that the pericardial transection device is in proximity to at least a portion of a nerve.

In one example, the at least one nerve proximity device is at least one nerve stimulation device. In one example, the at least one nerve stimulation device is located on the flexible catheter. In one example, the at least one nerve stimulation device is located adjacent the pericardial transection device 134. In one example, the at least one nerve stimulation device is located on the insertion tip 106. In one example, the at least one nerve stimulation device is located on a cutting surface. In one example, the at least one nerve stimulation device provides a proximity indication to at least a portion of a nerve, e.g., the phrenic nerve. The at least one nerve stimulation device can be an electrical lead or other electrical discharging device that can provide a visual indication to the user that the pericardial transection device is in proximity to at least a portion of a nerve, e.g., a breathing change of the patient when in proximity to the phrenic nerve.

In one example, the presently disclosed device discussed above can further comprise an optical channel in the transcatheter to accommodate a lens coupled to a fiber optic cable, optionally with a light source, e.g., an LED. In one example, the presently disclosed method further comprises obtaining visual information during accessing, traversal of the pericardial cavity 54, exiting and/or cutting, for example, using an optical channel in the transcatheter to accommodate a lens coupled to a fiber optic cable, optionally with a light source, e.g., an LED.

A medical kit, according to at least one embodiment of the present invention, comprises an elongated body 102 in the form of a guidewire or cannula, with an associated anchor device, such as any of the anchor devices described herein.

A medical kit, according to at least one embodiment of the present invention, comprises an elongated body 102 in the form of a guidewire or cannula, with an associated anchor device, such as any of the anchor devices described herein, and a catheter in which the elongated body and anchor are located or can be located for delivery to and retrieval from the patient, such as the pericardial cavity.

As a further embodiment of each of the above-described medical kits, such medical kits further include a medical device, such as a pericardial transection device, as described herein.

A medical kit, according to at least one embodiment of the present invention, comprises an elongated body 102 in the form of a guidewire or cannula, with an associated anchor device, such as any of the anchor devices described herein, and a multi-lumen catheter in which the elongated body and anchor are located or can be located in one or more lumens of the catheter for delivery to and retrieval from the patient, such as the pericardial cavity. In this or in further embodiments, other devices can also be located in the multi-lumen catheter, such as pericardial transection devices, multiple anchors, probes, sensors, guides (e.g., optical, fluorescent, etc.), and the like located in one or more lumens in the multi-lumen catheter.

While certain examples of the present disclosure have been illustrated with reference to specific combinations of elements, various other combinations can also be provided without departing from the teachings of the present disclosure. Thus, the present disclosure should not be construed as being limited to the particular exemplary examples described herein and illustrated in the Figures but can also encompass combinations of elements of the various illustrated examples and aspects thereof.

We claim:

1. A medical device for use in a medical procedure comprising:
a catheter having a central bore extending therethrough;
an elongated body comprising a proximal end and a distal end and configured for location in the central bore of the catheter;
at least one anchor coupled adjacent to the distal end of the elongated body for anchoring the distal end of the elongated body to a soft tissue, the at least one anchor having first and second configurations, wherein in the first configuration the at least one anchor is configured to avoid attachment to soft tissue and in the second configuration the at least one anchor is configured to secure itself relative to at least a portion of the soft tissue; and
a pericardial transection device configured for connection to a distal end of the catheter, wherein said pericardial transection device is configured for cutting pericardial tissue.

2. A medical device according to claim 1, wherein said pericardial transection device is movable relative to the at least one anchor and elongated body, such that the transection device may be slid relative to said elongated body on a path away or toward said at least one anchor.

3. A medical device according to claim 1 further comprising a piercing section for use in piercing the soft tissue, and wherein the at least one anchor is configured in the first configuration for location in the central bore adjacent the distal end of the catheter and configured to extend from the central bore in the second configuration.

4. A medical device according to claim 1 further comprising:
a tip located at the distal end of the catheter,
wherein the pericardial transection device is located on a side of the tip for extending laterally from a lengthwise axis of the tip.

5. A medical device according to claim 4 further comprising one or more supports configured to extend from the tip laterally from the lengthwise axis of the tip.

6. A medical device according to claim 1, wherein the at least one anchor comprises an expandable body configured for transitioning from the first configuration in a compressed state to an expanded state in the second configuration to thereby press the at least one anchor against the soft tissue.

7. A medical device according to claim 1, wherein the at least one anchor comprises one of:
an inflatable bladder, such that in the second configuration, the bladder is inflated to thereby press the at least one anchor against the soft tissue;
one or more collapsible and radially expandible arms for selectively contacting the soft tissue in the second configuration;
a helical screw ending in a distal end for inserting the at least one anchor into the soft tissue;
a clamp comprising two or more expandible arms configured to attach to the soft tissue; or
one or more protruding barbs configured to attach to the soft tissue.

8. A medical device according to claim 1, wherein the pericardial transection device is a scalpel, a mechanical cutting device, an electrode, an RF electrode, an electrosurgical device, a reversibly retractable knife blade, or combination of any two or more thereof.

9. A medical device according to claim 1, comprising a sheath configured for housing the at least one anchor prior to attaching the at least one anchor to the soft tissue.

10. A medical device according to claim 1, wherein at least a portion of the transection device is sterilized.

11. A medical device according to claim 1, comprising a probe associated therewith, wherein the probe is a nerve stimulating device or a nerve detecting device.

12. A medical device for use in a medical procedure comprising:
a catheter having a central bore extending therethrough;
an elongated body comprising a proximal end and a distal end and configured for location in the central bore of the catheter and movable within the elongated body;
at least one anchor coupled adjacent to the distal end of the elongated body for anchoring the distal end of the elongated body to the soft tissue, the at least one anchor having first and second configurations, wherein: in the first configuration the at least one anchor is configured for location in the central bore adjacent the distal end of the elongated body to avoid attachment to soft tissue, and in the second configuration the at least one anchor is configured to extend from the central bore to secure itself relative to at least a portion of the soft tissue;
a tip located at the distal end of the catheter;
a pericardial transection device configured for connection to a side of the tip for extending laterally from a lengthwise axis of the tip, wherein said pericardial transection device is configured for cutting pericardial tissue; and
one or more supports configured to extend from the tip laterally from the lengthwise axis of the tip.

13. A medical device according to claim 12, wherein said pericardial transection device is movable relative to the at least one anchor and elongated body, such that the transection device may be slid relative to said elongated body on a path away or toward said at least one anchor.

14. An elongated body according to claim 12, wherein the at least one anchor comprises an expandable body configured for transitioning from the first configuration in a compressed state to an expanded state in the second configuration to thereby press the at least one anchor against the soft tissue.

15. An elongated body according to claim 12, wherein the at least one anchor comprises one of:
an inflatable bladder, such that in the second configuration, the bladder is inflated to thereby press the at least one anchor against the soft tissue;
one or more collapsible and radially expandible arms for selectively contacting the soft tissue in the second configuration;
a helical screw ending in a distal end for inserting the at least one anchor into the soft tissue;
a clamp comprising two or more expandible arms configured to attach to the soft tissue; or
one or more protruding barbs configured to attach to the soft tissue.

16. An elongated body according to claim 12, wherein the elongated body is one of a guidewire or a cannula.

17. An elongated body according to claim 12, wherein the pericardial transection device is a scalpel, a mechanical cutting device, an electrode, an RF electrode, an electrosurgical device, a reversibly retractable knife blade, or combination of any two or more thereof.

18. An elongated body according to claim 12, comprising a sheath configured for housing the at least one anchor prior to attaching the at least one anchor to the soft tissue.

19. A medical device for use in a medical procedure comprising:
a catheter comprising an elongated body having a proximal end and a distal end and a central bore extending at least partially through the elongated body;
a guidewire comprising an elongated body located in said central bore of said catheter and spanning between the proximal and distal ends of the catheter;
an anchor coupled adjacent to a distal end of the guidewire for anchoring the guidewire to soft tissue, the at least one anchor having first and second configurations, wherein in the first configuration the at least one anchor is configured to avoid attachment to a soft tissue, and in the second configuration the at least one anchor is configured to secure itself relative to at least a portion of the soft tissue;
a tip connected to the distal end of the catheter;
a pericardial transection device located at a first position on a side of the tip for extending laterally from a lengthwise axis of the tip, said pericardial transection device comprising a cutting device configured to cut pericardial tissue; and
one or more supports configured located at one or more second locations on a side of the tip to extend from the tip laterally from the lengthwise axis of the tip.

20. A medical device according to claim 19, wherein said pericardial transection device is movable relative to the at least one anchor and elongated body, such that the transection device may be slid relative to said elongated body on a path away or toward said at least one anchor.
